(19) United States
(12) Patent Application Publication
(10) Pub. No.: US 2024/0058174 A1
(43) Pub. Date: Feb. 22, 2024

(54) SINGLE LOOP NON-SKIN ADHESIVE BANDAGE

(71) Applicant: Peter L. Levy, San Francisco, CA (US)

(72) Inventor: Peter L. Levy, San Francisco, CA (US)

(21) Appl. No.: 17/892,072

(22) Filed: Aug. 20, 2022

(51) Int. Cl. A61F 13/00; A61F 13/15; A61F 13/62

(52) U.S. Cl. CPC A61F 13/00038; A61F 13/00029; A61F 13/15268; A61F 13/15756; A61F 13/627; A61F 2013/00127

(57) ABSTRACT

A non-adhesive single wrap bandage with replaceable gauze for the wound area secured by expandable strip bandage with reusable bandage end coupling is disclosed. The non-skin adhesive skin bandage body has an array of perforations for skin aeration, risers, quick release gauze pads and mating end tabs for ease of bandage re-use.

Ser. No. 17/890,114

Oct. 30, 2022

### SPECIFICATION MODIFICATION

100—Sneeze goes into bag
110—Main Body
111—Right side
111a—Side Wing Which covers one cheek
112—Left side
112a—Side Wing Which covers one cheek
113—Base
114—Detachable edges
120—Tissue surfaces
130—Anti-bacteria

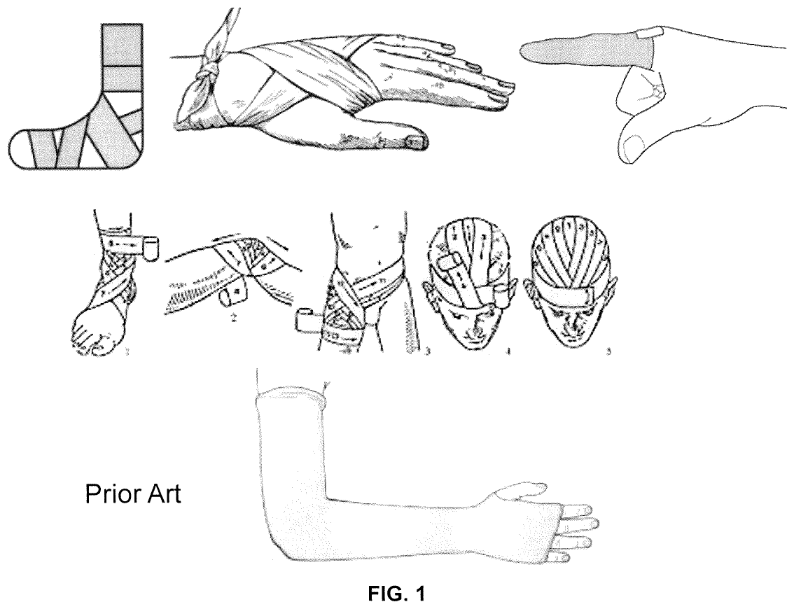

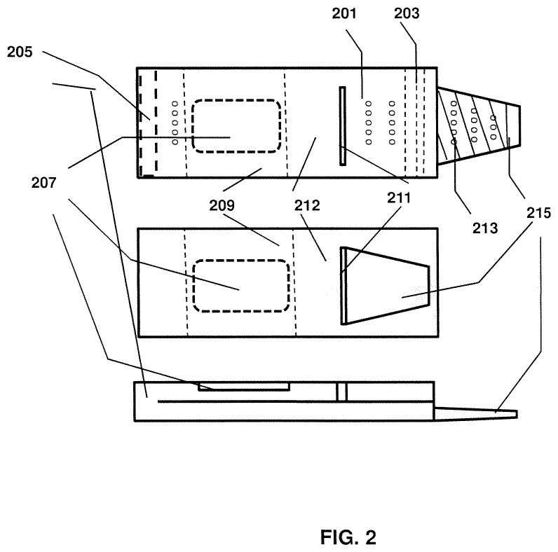

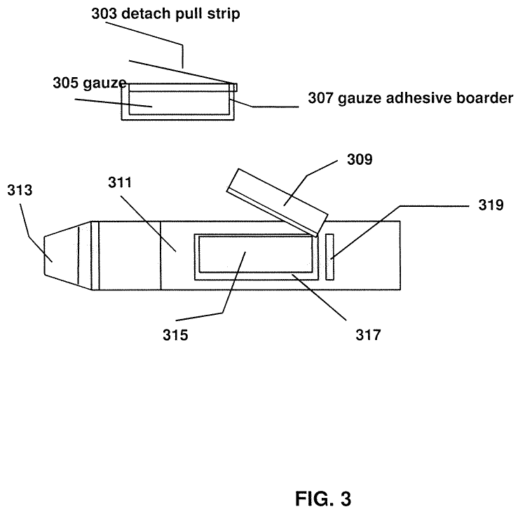

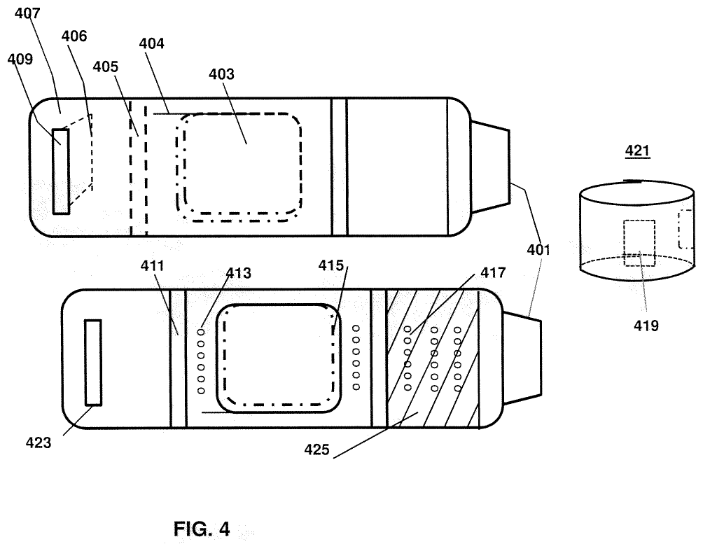

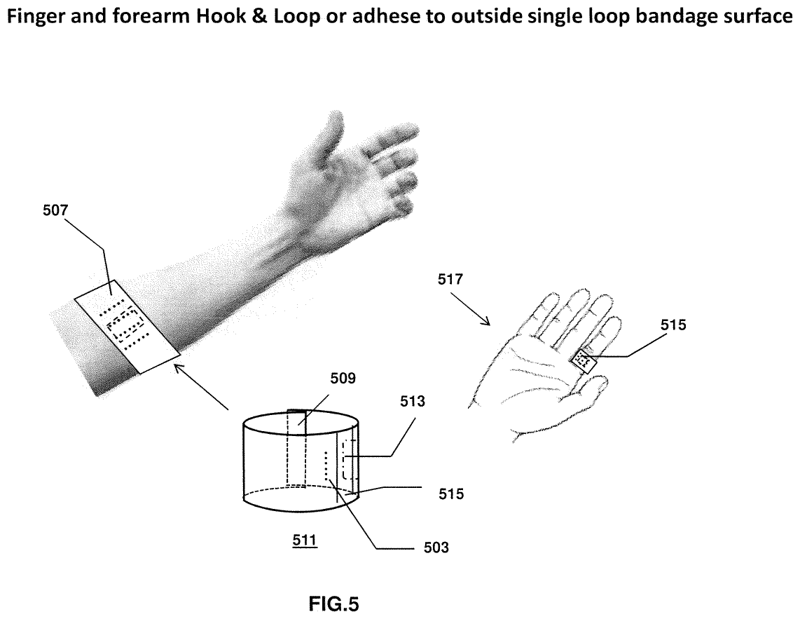

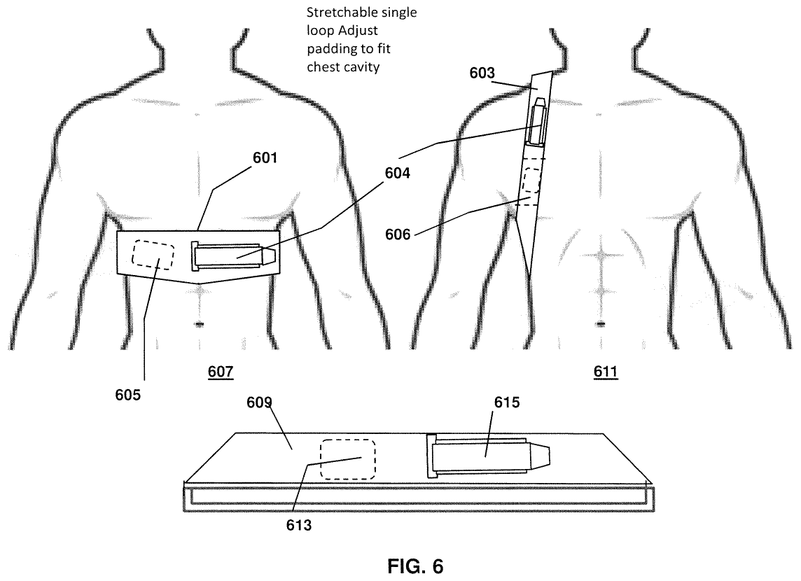

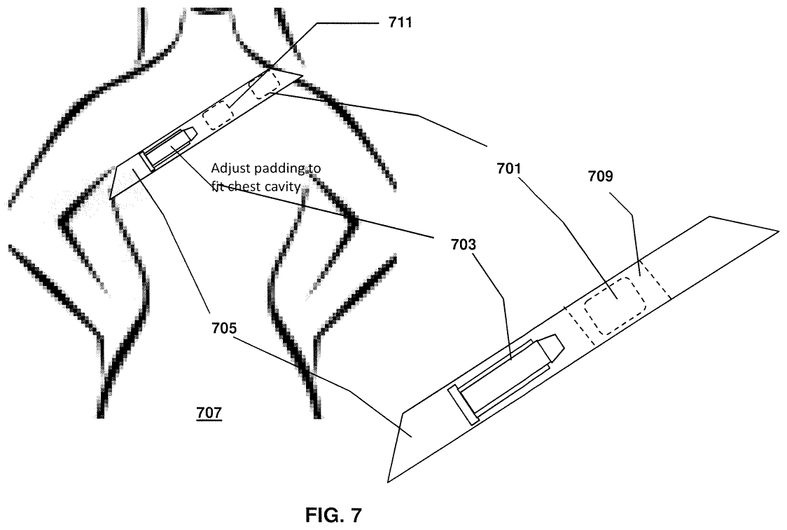

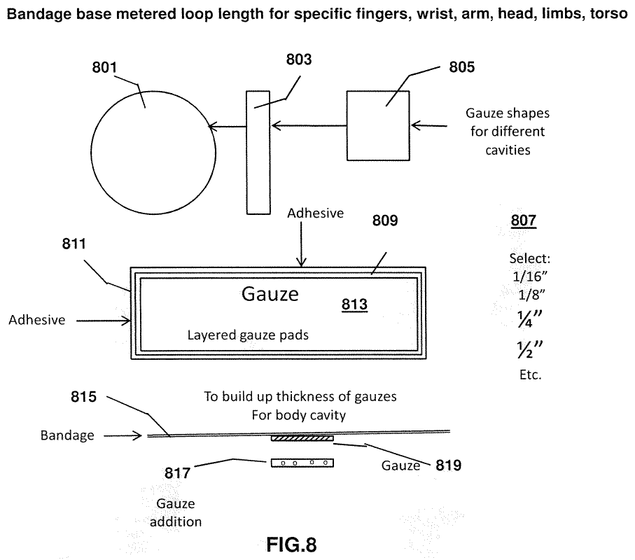

FIG. 1/1 is a full view of The Catch It Sneezing bag, container.

The Sneezing bag, container was constructed from plastic, tissue, tape, and anti-bacteria agent to trap sneeze, spit, cough on inside of bag and immediately kill pathogens. While hands stay clean.

### Actual Size

1. The main purpose of the disposable “Catch It” sneezing bags are to trap and absorb sneeze. To stop a Sneeze from traveling. To eradicate the probability of the spreading of germ and infectious bacteria.

2. The Sneezing Catching Container, when used will Catch WHATEVER SNEEZED. This introduces a more modern way to combat today's issues.

3. The Containers are made of plastic, paper, adhesives and structured to be used like a tissue.

4. The Sneezing bags Container are made so the nose and mouth are covered.

5. The Winged parts of the bag are used to wipe excess, mucus-like wetness left over from sneeze WITHOUT HANDS BEING CONTAMINATED.

6. Everything that would be in air in now trapped in bag, Sneezing container

This is a picture of what the Sneezing Containers will be catching.

This is a SNEEZE IN INCREMENTS OF 20 SECONDS

This is one of the way The Sneezing Container can be use at home or at your desk, like the double tissue.

New times calls for new ways.

What is claimed is:

1. A single loop stretchable non-skin adhesive bandage comprising:
a bandage with a base band providing a single loop of non-skin adhesive stretchable material about an appendage or body part having a surface wound, base band having two ends and two sides, a skin adjacent side and an opposite or band outside;
the two ends having a complementary ends for coupling the base band into a single loop;
the base band ends having re-attachable mating coupling at the base band ends to provide adjustable tension open-closing of the base band;
an attachable-detachable gauze coupled to the base band skin adjacent side;
the base band having air hole arrays and transverse mounted risers on the base band skin adjacent side;
whereby a single loop non-skin adhesive bandage can be repeatedly applied on a wound without skin adhesion or wound re-opening and having adjustable tension open-closing loop end coupling.

2. A single loop stretchable non-skin adhesive bandage as in claim 1 wherein the re-attachable mating coupling can be from a set of end couplings including a base band end tab with adhesive surface, inserting band end tab though a slot on the opposite base band end, folding the tab and coupling the tab to the base band outside surface through the slot on the opposite end, hook and loop, and non-skin adjacent side adhesive surface coupling.

3. A single loop stretchable non-skin adhesive bandage as in claim 1 further comprising the base band material with one or more segment(s) of non-stretchable material.

4. A single loop stretchable non-skin adhesive bandage as in claim 3 further comprising the base band with a first side facing a skin portion of a wound and having an non-stretchable segment attachable gauze pad with at least one gauze pad having a pull tab or gauze perimeter strip for detaching the gauze pad;

5. A single loop stretchable non-skin adhesive bandage as in claim 1 wherein the base band is made from stretchable material from a set of stretchable material including rubber, elastic, synthetic, nylon, cotton and stretchable blends.

6. A single loop stretchable non-skin adhesive bandage as in claim 1 wherein the base band of stretchable material is stretchable fabric blends from a set of fabric blends including cotton, polyester, satin, velvet, nylon, and rayon, rubber, latex neoprene, velvet, spandex, lycra, elastane, rayon, viscose, and microfiber.

7. A single loop stretchable non-skin adhesive bandage as in claim 1 further comprising the gauze with alternate polygonal shapes which are stackable and have discrete set of known thickness dimensions.

8. A method for single loop stretchable non-skin adhesive bandage comprising the steps of:
forming a bandage from a base band with a single loop of non-skin adhesive stretchable material about an appendage or body part having a surface wound, base band having two mating ends and two sides, a skin adjacent side and an opposite or base band outside;
providing the two base band ends each complementary to the other for coupling the base band into a single loop about the appendage or body part;
providing the base band ends with re-attachable mate coupling to have adjustable tension open-closing of the base band;
coupling an attachable-detachable gauze to the base band skin adjacent side;
mounting risers transverse to and on the base band skin adjacent side and providing air hole arrays on the base band;
whereby a single loop non-skin adhesive bandages can be repeatedly applied on a wound without skin adhesion or wound re-opening.

9. A method for single loop stretchable non-skin adhesive bandage as in claim 5 further comprising the step of providing re-attachable mate coupling from a set of adjustable coupling including tab with slot adhesive, hook and loop, and base band non-skin adjacent side adhesive surface coupling.

10. A method for single loop stretchable non-skin adhesive bandage as in claim 5 wherein the base band has one or more segment(s) of non-stretchable material.

11. A method for single loop stretchable non-skin adhesive bandage as in claim 8 further comprising the steps of providing the gauze pad with at least one gauze pad having a pull tab or gauze perimeter strip for detaching the gauze pad.

12. A method for single loop stretchable non-skin adhesive bandage as in claim 8 wherein the base band is made from stretchable material from a set of stretchable material including rubber, elastic, synthetic, nylon, cotton and stretchable blends.

13. A method for single loop stretchable non-skin adhesive bandage as in claim 8 wherein the base band of stretchable material is stretchable fabric blends from a set of fabric blends including cotton, polyester, satin, velvet, nylon, and rayon, rubber, latex neoprene, velvet, spandex, lycra, elastane, rayon, viscose, and microfiber.

14. A method for single loop stretchable non-skin adhesive bandage as in claim 8 further comprising the steps of providing the gauze with alternate polygonal shapes which are stackable and have discrete set of known thickness dimensions.
